(19) United States
(12) Patent Application Publication
(10) Pub. No.: US 2023/0372077 A1
(43) Pub. Date: Nov. 23, 2023

(54) MEDICAL IMPLANTS WITH NANO SURFACES

(71) Applicant: KINAMED, INC., Camarillo, CA (US)

(72) Inventors: William Ralph Pratt, Newbury, CA (US); Clyde Ronald Pratt, Santa Barbara, CA (US); Vineet Kumar Sarin, Moorpark, CA (US)

(21) Appl. No.: 18/200,703

(22) Filed: May 23, 2023

Related U.S. Application Data
(60) Provisional application No. 63/344,754, filed on May 23, 2022.

(51) Int. Cl. A61F 2/00; A61B 50/30

(52) U.S. Cl. CPC A61F 2/0095; A61B 50/30; A61L 2202/181; A61B 2050/314

(57) ABSTRACT

A medical implant or a method of packaging a medical implant is provided. In one embodiment, the medical implant may be packaged to facilitate remaining hydrophilic for an extended duration of time after packaging.

### TECHNICAL FIELD AND BACKGROUND

The present disclosure relates to medical implants and a method of improving the hydrophilic properties and organic compound-free state of a medical implant until the time of implantation.

The use of implants, both permanent and temporary, with titanium and titanium alloys surfaces is well known. Implants may be formed from titanium and titanium alloy bodies or may be formed from another base material, such as another metal or plastic, with a partial or complete titanium surface. To improve the bioactivity of the titanium implant surface or surfaces of the medical implant and, hence, enhance bone tissue ingrowth and/or resist microbial growth, the titanium surface of the medical implant is often treated, such as by roughening, by coating, or by a variety of chemical treatments.

More recently, implants have been formed with minute tubular structures (“nanotubes”) on the implant surface. These tubular structures typically have non-uniform cross-sections and can vary considerably from one nanotube to another. Nanotubes may have a predictable average pore size (effective inside diameter of a tube) measurable on the order of ten nanometers—and are collectively referred to as a nanotube surface.

Various attempts have been explored in order to delay or eliminate the deactivation of the surface but generally have failed to prevent the organic contamination sufficiently to assure that the titanium implant surface is active, namely substantially hydrophilic, at the time of surgery.

### SUMMARY

To preserve the hydrophilic properties of a medical implant with a titanium surface or component, including medical implants formed with titanium nanotubes, the medical implant is packaged after formation (which can include heat treating) in a manner as to prevent subsequent surface contamination. Further, the packaging is configured to assure that little or no air is present within the packaging, and that the packaging is configured to be sufficiently robust to maintain the organic-free and carbon gas-free atmosphere through the packaging and sterilization processes, as well as product storage, shipment, and delivery to the operating room.

In one embodiment, a packaging process uses a package that can maintain a low pressure, such as a partial pressure vacuum, during its time of use and which can be processed so that little or no organic material or carbon gas is present when the package is sealed with the implant inside. The active implant surface can retain its hydrophilicity for at least 3 years from the time of processing, so that a water drop measurement will show a water contact angle of less than 90 degrees.

In another embodiment, a packaging process for an implant with a titanium implant surface uses a package that can maintain a low pressure, such as a partial pressure vacuum, during its time of use and which can be processed so that little or no organic material is present when the package is sealed with the implant inside.

In one aspect, prior to placing the implant in the package, the implant is selected so that it has a titanium implant surface with a nanotube surface that is hydrophilic with a water contact angle of less than 5 degrees at or immediately after formation.

In one aspect, the implant is placed in and sealed in the package in a manner so that (a) after three years the nanosurface remains hydrophilic, or (b) after one year the water contact angle is less than 30 degrees or (c) after 120 days the water contact angle is (i) optionally less than 20 degrees, (ii) optionally less 10 degrees, and (iii) optionally less than 5 degrees.

In another aspect, the implant is placed in and sealed in the package in a manner so that (a) after three years the nanosurface retains at least 10% of hydrophilicity, (b) after one year retains at least 20% of hydrophilicity, or (c) less than one year but after at least 120 days retains 80% of hydrophilicity, optionally 90% of hydrophilicity, or optionally about 100% of hydrophilicity.

In another embodiment, a packaging process uses a package that can maintain a low pressure, such as a partial pressure vacuum during its time of use and which can be processed so that little or no organic material or carbon gas is present when the package is sealed with the implant inside. Prior to placing the implant in the package, the implant is selected so that it has a nanotube surface with a water contact angle of less than 5 degrees at or immediately after formation, which can include heat treatment. The implant is then placed in and sealed in the package in a manner so that the water contact angle increases at an average rate of less than 1/10th of a degree per day.

In one aspect, in any of the above methods, the method may be enhanced by including within the package, or in the packaging itself, a scavenger, such as an absorber or adsorber, that specifically and actively attracts and captures the organic material, or contributors such as carbon gas, to reduce and eliminate any remaining available organic material, or contributors such as carbon gas, within the sealed package and which protects the implant from subsequent contamination prior to its implantation.

In another or further aspect, prior to placement in the package, the implant and/or the inside of the package is treated with UV light, such as UVC light, to substantially remove any organic compound that may have attached to the nanotubes prior to placement of the implant in the package and/or on the inside of the package.

In another or further aspect, after placement in the package, the implant is exposed to UV light, such as UVC light, to remove substantially any organic compound that may have attached to the nanotubes.

In another or further aspect, prior to placement in the package, the package is filled with an inert gas, such as Argon or Nitrogen, or another carbon displacing gas to displace any gases, including carbon gases in the package, so that the implant is inserted into the inert gas.

In another embodiment, the packaging process uses a package that can maintain a low pressure, such as a partial pressure vacuum, during its time of use and which can be processed so that little or no organic material, including contributors such as carbon gas, is present when the package is sealed. The process may be bolstered by including within the packaging container, or in the packaging itself, a scavenger, such as an absorber or adsorber, that specifically and actively attracts and captures the carbon gas to reduce and eliminate any remaining available carbon gas within the sealed package and which protects the article from subsequent contamination prior to its implantation.

In yet another method of handling a medical implant, where the implant includes a titanium implant surface with a plurality of TiO2 nanotubes formed thereon, the method includes providing a sealable package and, depending on additional steps that may be performed, placing the medical implant in the sealable package within 12 hours, or within about 6 hours, or within about 2 hours of formation i.e., after the implant has been formed with the TiO2 nanotubes and optionally annealed. Once placed in the package, and depending on additional steps that may be performed, the implant is sealed in the sealable package to form a sealed package within less than about 1 hour, optionally less than about 30 minutes, and optionally less than about 2-3 minutes after the last post-formation treatment process and/or placing the medical implant in the sealable package.

In one aspect, the method further includes removing the air contained inside the sealable package after placing the medical implant in the sealable package to form a low organic or low carbon gas atmosphere or an organic-free or a carbon gas-free atmosphere in the sealed package.

In another or further aspect, the method further includes removing the air contained inside the sealable package prior to placing the medical implant in the sealable package.

In another aspect, the method includes providing a sealable package that is sufficiently robust to maintain an organic-free or a carbon gas-free atmosphere in the sealed package through the packaging processes and product storage, shipment, and delivery to an operating room.

In another aspect, the method includes sterilizing the sealed package, with the sealable package being sufficiently robust to maintain an organic-free or a carbon gas-free atmosphere in the sealed package through the sterilization process.

In another aspect, the method includes providing a sealable package that is capable of maintaining a low or partial pressure vacuum during its time of storage prior to implantation.

For example, in any of the above, the sealable package may be formed from a flexible heat sealable synthetic material, such as a material selected from the group consisting of Polyethylene (PE). Polyvinylidenchloride (PVDC), Polypropylene (PP). Polyvinylchloride (PVC). Polyester (PET), and Polyamide (PA) laminated with a metal foil, such as aluminum foil. Alternatively, in any of the above methods, the package may be a rigid material, such as metal, glass, or a plastic. The term rigid is used broadly and intended to cover any package that can hold its own shaped without being pressurized.

In yet other aspects, the method further includes providing a scavenger, such as an absorbent or adsorbent, to actively attract and capture any carbon gas present in the sealable package to reduce and/or eliminate any available organic or carbon gas within the sealed non-porous package. As noted below, in other configurations, the package may be gas porous.

In yet further aspects, the method includes placing the absorbent or adsorbent in the sealable package prior to placing the medical implant in the sealable package.

In other aspects, after sealing the implant and the scavenger in the sealable package and after a period of time after sealing (to allow the scavenger to reduce and/or eliminate any available organic material or carbon gas within the sealed package), the method further includes sealing the scavenger in a portion of the heat sealed package to form a separate compartment from a main compartment enclosing the medical implant with a path for gaseous exchange between the two compartments.

In another aspect, providing a sealable package includes providing a sealable package with a main compartment for holding the medical implant and a secondary compartment for holding the absorbent or adsorbent.

According to another method of handling a medical implant, the method includes providing a sealable package, displacing the organic material from the nanotubes formed on the medical implant, placing the medical implant in the sealable package within 2 hours after displacing the organic material from the implant, and then sealing the implant in the sealable package to form a sealed package within about 60 to 2 minutes after placing the medical implant in the sealable package to form a low organic material or low carbon gas atmosphere or an organic-free or carbon gas-free atmosphere in the sealed package.

For example, the displacing includes exposing the medical implant to UV light and/or exposing the interior of the package to UV light.

In one aspect, the method further includes sterilizing the sealed package.

In a further aspect, the method further includes providing an absorbent or adsorbent that actively attracts and binds any carbon gas present in the sealable package to reduce and/or eliminate any available carbon gas within the sealed package, and removing the absorbent or adsorbent before sterilizing.

For example, the sealable package may include a main compartment for holding the medical implant and a secondary compartment for holding the absorbent or adsorbent. Optionally, the secondary compartment may be removable from the main compartment without unsealing the main compartment.

According to another method of handling a medical implant, the method includes providing a sealable package with a carbon absorber or carbon adsorber, and optionally a carbon repellant, integrated into the package, placing the medical implant in the sealable package, and then sealing the implant in the sealable package to form a sealed package to produce and maintain a carbon gas-free atmosphere in the sealed package.

In one aspect, the sealable package comprises a lamination of a plurality of layers, with one or some of the layers forming the carbon absorber or adsorber or repellant. For example, the innermost layer may form the carbon absorber or adsorber or repellant. Alternately the absorber or adsorber or repellant may be incorporated into one of the layers forming the package. A suitable carbon absorber may be provided in the form of a layer or other component of the package. A suitable repellant may include a graphene layer or a layer incorporating graphene. In this manner, such a repellant layer acts as a molecular sieve or filter integrated into the packaging.

In another embodiment, a method of packaging a titanium implant includes flushing the medical implant in an air tight container with an inert gas, which includes a UVC light source and an absorber or adsorber. The gas flush reduces the amount of organic material, including contributors such as carbon gas, to start off with, and the UVC breaks the bonds that may be occurring between the titanium implant and organic material, and the absorber is actively scavenging the carbon gas from the enclosed atmosphere.

In any of the above, the method may further include immediately coating the active titanium surface with sodium chloride molecules such as when the coating is achieved by heating NaCl to 700 degrees centigrade and passing an argon gas over the surface of the NaCl to create a flow of NaCl molecules, produced by sublimation, which are deposited upon the titanium surface to protect the surface from organic contamination.

In any of the above, the method may include immediately coating the nanotubes with an appropriate polymer coating, dissolved in H2O or alcohol, so that the nanotubes are protected from organic contamination.

In any of the above, the method may further include storing the sealed package in a larger air tight container. For example, the larger air tight container may include a scavenger, such as an absorbent or adsorbent, contained therein wherein the ambient atmosphere around the sealed package has an atmosphere with the carbon gas removed.

In another embodiment, a medical implant is provided that is packaged and handled in accordance with any of the above methods.

In another embodiment, a medical implant is provided that has been handled and/or stored for less than one year and at least 120 days and that has retained at least 80% of hydrophilicity, 90% of hydrophilicity, or about 100% of hydrophilicity.

In another embodiment, a medical implant is provided that has a medical implant body with a titanium implant surface with an initial water contact angle of less than 5 degrees immediately after formation and pre-packaging and a post packaging water contact angle, where the post packaging water contact angle covers a packaging period of less than 1 year and at least 120 days, and which post packaging water contact angle is less than 30 degrees, optionally, less than 20 degrees, and optionally less than 10 degrees.

### BRIEF DESCRIPTION OF THE DRAWINGS

FIG. 1 is a side elevation view of a medical implant that may incorporate a nanotube surface;

FIG. 2 is a similar view to FIG. 1, with the fasteners removed;

FIG. 2A is an enlarged schematic view of the nanotube surface of the medical implant of FIG. 1, which forms a medical implant surface:

FIG. 3 is a plan view of another embodiment of a medical implant; and

FIG. 4 is a schematic drawing of the medical implant package.

It is to be understood that the drawings are for purposes of illustrative purposes only and do not represent limitations to the scope of the disclosure.

### DETAILED DESCRIPTION

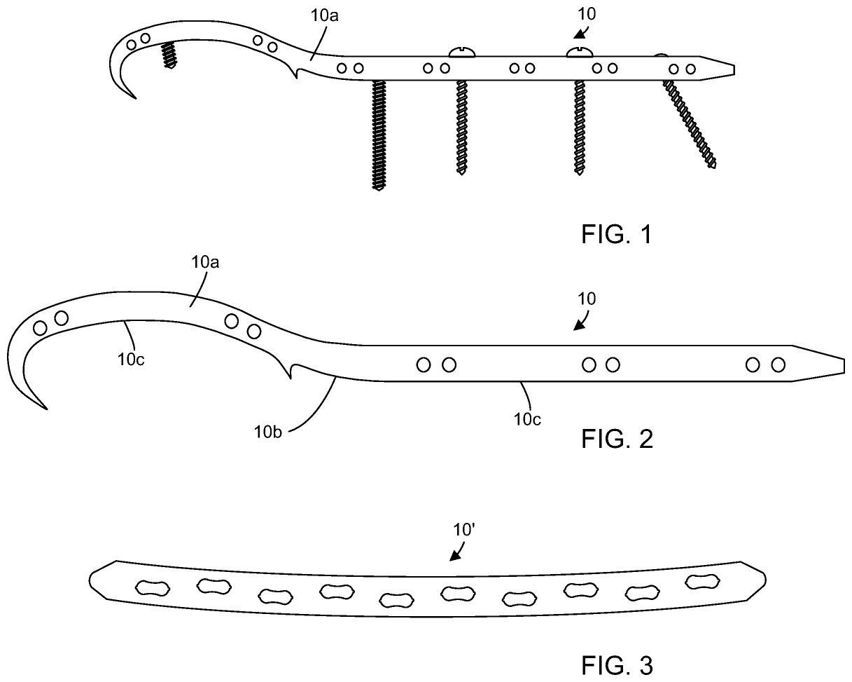

Referring to FIG. 1, the numeral 10 generally designates a medical implant formed from titanium or titanium alloy or other base material, such as another metal, including chromium cobalt, or a plastic, and which has a titanium or titanium alloy implant surface that may be formed with a nanotube surface, which is suitable for implant with bone. Such nanotube surfaces, in a crystalline state, either anatase or rutile, are highly hydrophilic at the time of processing. The combination of a specified diameter, appropriate phase, either anatase or rutile, or a mixture of the two, and a high surface energy condition, create an effective anti-microbial surface that resists colonization of the surface by bacteria. This “activated” surface will also stimulate the early formation of bone cells upon the surface. However, as noted above and below, the activity (hydrophilicity) of this surface can be degraded by organic material contamination, such as by carbon dioxide gas.

In the illustrated embodiment, the implant 10 is illustrated as a bone plate, such as a contoured trochanteric plate. Referring to FIG. 3, an alternate bone plate 10′, is curved to preferentially match the femoral bow. Hereinafter for ease of reference, reference will be made to the implant 10, although it should be understood that the processes and methods described herein equally apply to the implant 10′. Thus, the illustrated forms of the implant are for illustrative purposes only, and it should be understood that the packaging as well as the post formation processes described herein may be used for other implants, including permanent implants as well as temporary implants, and for a variety of implants, including total joint implants, partial joint implants, as well as catheters, screws, meshes, stents, housings, spacers, connectors, rods, pins, wires, bone replacements, tissue scaffolds, fixation devices, nails, trans-cutaneous interfaces, and anchors and any other types of implants, including a group of implants, designed for implantation which are fully or partially covered by titanium or an alloy of titanium surface.

As noted above, the implant 10 is formed from a base material, such as titanium or titanium alloy, chromium cobalt, or a plastic, which is formed into the desired shape or body 10a, and is provided with a titanium implant surface 10b, which optionally covers the entire implant surface of the body 10a. The titanium implant surface 10b may be formed with one or more TiO2 nanotube surfaces 10c, each with a plurality of nanotubes 10d, using a number of different processes. For example, TiO2 nanotubes may be formed by anodizing or other processes, such as 3D printing. The implant 10 may also be formed without nanotubes, and instead may be prepared by roughening, plasma spraying, or chemically treating the titanium implant surface.

When formed with nanotubes, the diameter, length, and density of the nanotubes may vary. The combination of the diameter (as conventionally assessed), appropriate phase (e.g., either anatase or rutile, or a mixture of the two), and an active surface energy condition, create an effective anti-microbial surface that resists colonization of the surfaces by bacteria. This “activated” surface will also stimulate the early formation of bone cells upon the surface.

In one example, the implant is selected with a nanotube surface with a heterogeneous mixture of diameters with a characteristic mean diameter in a range of about 3 nm to about 300 nm, optionally about 5-120 nm, and optionally about 20-100 nm, and optionally about 80 nm. Further, the nanotubes may be free standing, though they may have other arrangements. The nanotubes may be fabricated to have a length ranging from about 1 μm to about 100 μm, and have a pore size that range from about 3 nm to about 100 nm, optionally about 5 nm to 50 nm, and optionally about 10 nm.

Further, the implant surface may have two or more nanotube surfaces each with different heights, diameters, and/or densities. For example, one area may have nanotubes with a first height, and a second area having nanotubes with a second height different than the first height. In another example, one area may have nanotubes with a first density, and a second area having nanotubes with a second density different than the first density. In yet another example, one area may have nanotubes with a first diameter, and a second area having nanotubes with a second diameter different than the first diameter.

The TiO2 nanotube surfaces may also undergo an additional crystallization annealing process through exposure to a crystallization annealing temperature, after the anodization process has been performed, in order to form an anatase or rutile or a mixed phase of TiO2. This crystallization annealing temperature may generally be performed at a temperature (Ti) of about 200° C. to 600° C. about 250° C. to 450° C., and optionally about 380° C.

In general, three known methods can be used for preparing TiO2 nanotubes: the “template” method, the “hydrothermal (solvothermal)” method, and the “electrochemical anodization” method. For the template method, TiO2 nanotubes have a larger inner diameter and thicker tube wall, and their morphology is restricted by the template. With the hydro-thermal (solvothermal) method, TiO2 nanotubes have small tube diameters, thin tube walls, and their morphology is also difficult to control. Of these methods, the anodic oxidation method displays the simplest operation process and has the advantages of vertical arrangement and highly ordered nanotube arrays. See second paragraph in: https://www.researchgate.net/publication/348675726_Highly_Ordered_TiO2_Nanotube_Arrays_with_Engineered_Electrochemical_Energy_Storage_Performances.

For more details of suitable examples of nanotube structures and processes for making the nanotube surfaces, reference is made to: U.S. Pat. No. 8,414,908, entitled COMPOSITIONS COMPRISING NANOSTRUCTURES FOR CELL, TISSUE AND ARTIFICIAL ORGAN GROWTH, AND METHODS FOR MAKING AND USING SAME; U.S. Pat. No. 9,273,277, entitled COMPOSITIONS COMPRISING NANOSTRUCTURES FOR CELL, TISSUE AND ARTIFICIAL ORGAN GROWTH, AND METHODS FOR MAKING AND USING SAME; U.S. Pat. No. 9,844,657, entitled COMPOSITIONS COMPRISING NANOSTRUCTURES FOR CELL, TISSUE AND ARTIFICIAL ORGAN GROWTH, AND METHODS FOR MAKING AND USING SAME; U.S. Pat. No. 9,149,564, entitled ARTICLES COMPRISING LARGE-SURFACE-AREA BIO-COMPATIBLE MATERIALS AND METHODS FOR MAKING AND USING THEM; U.S. Pat. No. 9,867,903, entitled ARTICLES COMPRISING LARGE-SURFACE-AREA BIO-COMPATIBLE MATERIALS AND METHODS FOR MAKING AND USING THEM; U.S. Pat. No. 9,623,151, entitled BIOMATERIALS AND IMPLANTS FOR ENHANCED CARTILAGE FORMATION, AND METHODS FOR MAKING AND USING THEM; U.S. Pat. Pub. App 2018/0297839 entitled ARTICLES COMPRISING NANO-MATERIALS FOR GEOMETRY-GUIDED STEM CELL DIFFERENTIATION AND ENHANCED BONE GROWTH; U.S. Pat. No. 10,149,921, entitled PRODUCTS OF MANUFACTURE HAVING TANTALUM COATED NANOSTRUCTURES; U.S. Pat. No. 9,376,759, entitled COMPOSITIONS, METHODS AND DEVICES FOR GENERATING NANOTUBES ON A SURFACE; and U.S. Pat. No. 10,857,575 entitled SHELF-LIFE-IMPROVED NANOSTRUCTURED IMPLANT SYSTEMS AND METHODS, which are all incorporated by reference herein in their entireties.

Optionally, when selecting the implant, the method includes selecting an implant with the lowest water contact angle as possible, and then maintaining the water contact angle and optionally keeping the water contact angle below a maximum water contact angle until implantation. “Water contact angle” is a commonly understood term to refer to the angle at which a drop of distilled water forms with a flat surface on which the drop is lying. There are numerous methods of measuring the angle, including the use of optical comparators, but for the purpose of this disclosure any of the conventional methods may be used to determine the water contact angle. Typically a hydrophilic surface is regarded as having a water contact angle of less than 90 degrees, while a hydrophobic surface is regarded as having a water contact angle of greater than 90 degrees. A surface with a perfect hydrophilic surface (often referred to as “super hydrophilic”) has a zero water contact angle.

As will be more fully described below, the packaging alone or in combination with one or more pre-packaging and/or post-packaging treatment processes described herein are believed to help the implant remain hydrophilic even after 3 years after packaging. Further, it is believed that after the implant is placed in and sealed in the package in the manner described herein that after one year (after packaging) the water contact angle is less than 30 degrees or after 120 days the water contact angle is optionally (a) less than 20 degrees, (b) less 15 degrees, (c) less than 10 degrees, and (d) less than 5 degrees. It is also believed that after the implant is placed in and sealed in the package in the manner described herein that (a) after three years after being packaged, the nanosurface retains at least 10% of hydrophilicity, (b) after one year retains at least 20% of hydrophilicity, or (c) less than one year but after at least 120 days retains 80% of hydrophilicity, optionally 90% of hydrophilicity, or optionally about 100% of hydrophilicity.

The expression of the % of hydrophilicity refers to the percentage of what would be considered perfect hydrophilicity—in other words a surface with a 0 degree water contact angle, therefore, retaining about 100% of hydrophilicity means it has retained about a 0 degree water contact angle. In contrast, because the water contact angle range of hydrophilicity is from 0 to 89.9, with an increasing water contact angle indicating a less hydrophilic surface, retaining 90% of hydrophilicity can be expressed by the equation:

100 times (X−Y)/X where is X is the range of hydrophilicity and Y is the current hydrophilicity, both as measured in degrees of water contact angle.

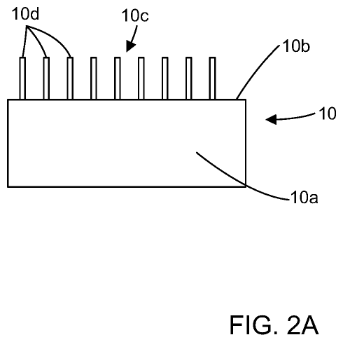

As best seen in FIG. 2, the implant 10 may include a nanotube surface 10c covering at least a portion of the surface of the implant 10. The nanotube surface 10c is formed from a plurality of nanotubes. While only one nanotube surface 10c with nanotubes are shown in FIG. 2A, it will be understood that the number of nanotubes may vary in number, size, orientation and spacing.

As noted above, when selecting the implant, an exemplary implant may have a nanosurface with a plurality of TiO2 nanotubes selected with a heterogeneous mixture of diameters with a characteristic mean diameter in a range of about 3 nm to about 30) nm, optionally about 5-120 nm, optionally about 20-100 nm, and optionally about 80 nm. Further, the method includes selecting an implant with a water contact angle of less than 3 degrees, and optionally less than 1 degree. The packaging process described below is believed to be able to maintain the water contact angle of the implant to below a maximum angle of 30 degrees, below 20 degrees, below 10 degrees, or below 5 degrees after four months (120 days) after being sealed in the package. Alternately, the packaging process described below is believed to be able to maintain the water contact angle of the implant in a manner so that the water contact angle degrades at an average rate of less than 1/10th of a degree per day.

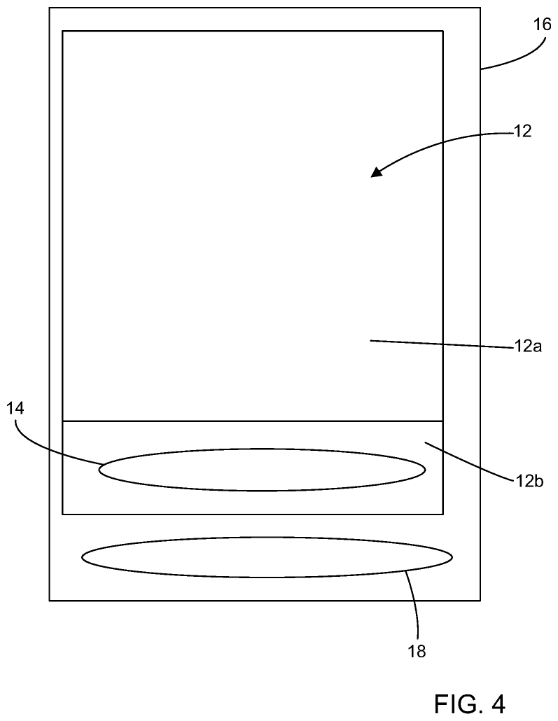

In order to preserve the hydrophilicity of the nanotube surface(s) 10c, the process described herein isolates the implant from organic contamination, optionally from immediately after formation until its implantation. Referring to FIG. 4, to preserve the hydrophilic properties of the implant 10, which as noted is formed with a titanium nanotube surface 10a, the implant 10 is sealed in the package 12 after forming the implant 10 with its nanotube surface or surfaces and after annealing (and the nanotubes are crystalized in the anatase or rutile or a mixture of the two phases).

For example, the implant 10 is scaled in the package 12 within less than 2 hours or less than 1 hour or less than 30 minutes after forming the implant 10—in other words, the implant 10 may be sealed in the package 12 directly after the formation process, optionally including after being removed from the chamber that provides the anodizing process to form the nanotube surface. In this manner, the implant 10 may be protected immediately after formation (e.g., within in 30 minutes after the final step of forming—for example the annealing step). As will be more fully described below, the implant 10 is packaged in a manner as to prevent subsequent contamination until the implant is implanted. Further, as will be more fully described below, the medical implant may be subject to post-formation and pre-packaging treatment or post formation and post packaging treatments. Hence, the time periods described below may vary depending on the number and/or type of post formation and pre-packaging treatments or post formation and post packaging treatments.

In one example, where the implant is subject to post formation treatment, such as the UV treatment or coating processes described below, the time period after formation within which the implant is placed in the package may in a range of about 2 to 12 hours, about 2 to 8 hours, or optionally 2 to 4 hours.

In another embodiment, a packaging process uses a package that can maintain a low pressure, such as a partial pressure vacuum, during its time of use and which can be processed so that little or no organic material is present when the package is sealed with the implant inside. Prior to placing the implant in the package, the implant is selected so that it has a nanotube surface with a water contact angle of less than 10 or 5 degrees at or immediately after formation. The implant is then placed in and sealed in the package in a manner so that the water contact angle increases at an average rate of less than 1/10th of a degree per day.

Referring to FIG. 4, the implant 10 is inserted into and fully enclosed in the package 12, which may be formed from a flexible heat sealable package, such a polymer material, including Polyethylene (PE), Polyvinylidenchloride (PVDC), Polypropylene (PP), Polyvinylchloride (PVC), Polyester (PET), and Polyamide (PA), and optionally laminated with a metal foil, such as an aluminum foil, to form a gas barrier in the packaging. In this manner, the packages may be manufactured with a flat configuration and thus easily stored that way.

In one embodiment, once the implant 10 is placed into the package 12, the package 12 is then hermetically sealed. The package 12 may be sealed using a sealer where substantially all air is removed from the package 12 before sealing, such as heat sealing.

In one embodiment, the package 12 may be processed in a gas flushed glove box apparatus wherein the pouches are opened and the product inserted, so it is does not have air entering the package in the first place.

In an alternate embodiment the package may be rigid—and formed from a non-porous metal, glass, or a plastic material. The term “rigid” as used herein is used broadly to include a package that can retain its shape without being inflated or pressurized.

Once placed in the package 12, for example, the implant 10 is sealed therein after air is removed from the package to form a low or partial vacuum pressure sealed package, for example, by removing air to reduce the pressure in the package. Further, the package material and sealing process is selected so that it can maintain the low pressure post sealing through to use.

In one embodiment, the package is placed in a chamber wherein the product is put into the package where it is open to atmosphere and then flushed, followed by the package being positioned for sealing. In this manner, the chamber is gas flushed and then followed by sealing the package.

Consequently, the packaging and sealing process is selected such that it can assure that little or no air is present within the packaged implant and that the package is sufficiently robust to maintain a low organic or carbon atmosphere or organic-free or carbon gas-free atmosphere (as more fully described below) through the packaging and sterilization processes, as well as product storage, shipment, and delivery to the operating room. To assure little or no air is present and it is sufficiently robust, the package 12 may be selected from a group of synthetic materials consisting of any of the above listed polymers, which is laminated with a metal foil, such as aluminum foil, and/or the air drawn from the package may be performed with a low suction apparatus to avoid damaging the package.

Alternately or in addition, as described below, the package 12 may be formed from a primary package, such as described above, and a secondary outer package, as noted below, to increase its ability to maintain the low organic or carbon atmosphere or organic-free or carbon gas-free atmosphere through the packaging and sterilization processes, as well as product storage, shipment, and delivery to the operating room.

Prior to placing the implant 10 in the package 12, the package 12 may be flushed with an inert gas, such as Argon or Nitrogen, or another carbon displacing gas to displace or remove possible contamination in the package prior to placing the implant 10 into the package 12. The injection or flushing the package may also be done after the implant is placed in the package.

In another aspect, a pre-conditioning process may also be used to convert carbon gases into other forms in carbon dioxide. For example, the package may include a catalyst or reactant, such as platinum, to convert carbon monoxide to carbon dioxide and make that potential contribution of carbon available for capture by the CO2 absorber or adsorber described below.

Optionally, the package 12 may include a carbon gas scavenger 14 absorber (carbon gas enters into liquid or solid) or adsorber (carbon gas attaches to surface) that bonds with any carbon gas that enters the package 12 either before, during, and/or after placement of the implant 10 into the package 12. By specifically and actively attracting and binding to any carbon gas that may be present or enter into the package, the scavenger reduces and eliminates any remaining available carbon gas within the sealed package container and protects the implant from subsequent contamination prior to its implantation. Furthermore, the scavenger attracts and captures carbon gas that may molecularly diffuse through the walls of the package prior to the use of the implant. As would be understood when using rigid packages, the concern about the packaging being crushed after air is removed from inside the container is avoided.

For example, a suitable scavenger may comprise Zeolite or soda lime or metal-organic frameworks (MOFs) or other commercially available carbon absorbers and adsorbers.

As another example, the carbon gas scavenger 14 may chemically interact with CO2 gas and incorporate it into a solid. In yet another example, the carbon gas scavenger 14 may be a desiccant that mechanically filters out and traps carbon bearing gases at a molecular level. This type of desiccant-based approach may correspond to a molecular sieve configuration. In some configurations, the reaction rate for various types of carbon gas scavengers 14 may be substantially similar to each other.

Referring again to FIG. 4, the package 12 may have a main location or compartment 12a, where the implant is placed, and a secondary location or compartment 12b, which is in fluid communication with the main compartment via a path, with the carbon gas scavenger 14 placed or located in the secondary compartment. The package is then sealed. In this manner, the scavenger can capture the carbon gas from the air in the main compartment.

The path may then be closed or the separation between the two locations may be formed after the package is sealed and the scavenger has had time to attract and bond with or absorb the carbon gas.

For example, the two separate compartments may be formed by heat-sealing the package along a seam that separates the package into the two areas, for example, after the package is initially sealed.

Additionally, the secondary compartment may be separated from the package in a manner to maintain the seal of the main compartment. Thus, the scavenger may be confined to a particular location within the package and, further, can be subsequently sealed off from the main compartment without compromising the continuity of protection of the implant remaining in the main compartment.

Furthermore, as noted, the isolated scavenger may be physically detached from the main compartment and removed from the product configuration. This would allow the scavenger to have a period of activity to perform its function, after which it can be removed before creating later complications, such as presenting a potential safety hazard to the end user.

Therefore, once the package 12 is sealed, the package and the implant contained therein may be sterilized after the scavenger is removed.

In another embodiment, the package may incorporate a CO2 scavenger layer in or integrated in the packaging layer that forms the package (e.g., in the package laminate itself) or a CO2 “resistant” or “repellant” layer in or integrated into the packaging layer. For example, one suitable CO2 resistant or repellant layer is an aluminum layer.

Optionally, as noted above, the implant 10 may be subjected to post formation treatment before placement in the package. In one embodiment, prior to placement in the package 12, the implant 10 may be exposed to ultraviolet (UV) light of a specific range of wavelengths and energy, for example UVC light. The UV exposure is applied for sufficient time to displace the organic material from the surface and thus restore a significant amount of any lost hydrophilicity prior to packaging. For example, the UV exposure may be applied for about 60 to 10 minutes, for about 40 to about 15 minutes, or optionally for about 20 minutes.

For example, the implant 10 may be placed in an enclosure with a plurality of UV light sources, such as UVC lamps. Optionally, the enclosure may be filled with an inert gas, such or Nitrogen or Argon, or another carbon displacing gas. Suitable UV light may include, for example, UVC light. For more details of an example of other suitable UV treatment, reference is made to U.S. Pat. No. 10,857,575, entitled SHELF-LIFE-IMPROVED NANOSTRUCTURED IMPLANT SYSTEMS AND METHODS which is incorporated by reference in its entirety herein.

In another embodiment, after placement in the package 12, but before sealing, the implant 10 may be exposed to ultraviolet (UV) light, for example UVC light. Similarly, the UV exposure is applied for sufficient time, such as described above, to displace carbon atoms from the surface and thus restore a significant amount of any lost hydrophilicity prior to packaging.

In this embodiment, the implant 10 and unsealed the package 12 may be placed in an enclosure with a plurality of UV light sources, such as UVC lamps. Further, the enclosure may be filled with an inert gas, such as Argon or Nitrogen, or another carbon displacing gas to displace any carbon gas.

Additionally, post formation, but prior to packaging, the implant may additionally include an application of a sodium chloride solution or a surfactant, immediately after the annealing process and then dried. This would provide a further protective layer directly on the article that can be conveniently washed with water, such as deionized water, by the end user at time of use.

Optionally, as noted above, the packaging process may include a system of packages, including double packaging. For example, after sealing, one or more packages 12 may be placed and stored in a larger, outer air tight container 16 with its own scavenger 18 (absorbent or adsorbent) contained therein so that the ambient atmosphere around the sealed package(s) 12 has a modified atmosphere with the organic material, as well as contributors such as carbon gas, removed, thus precluding the possibility of carbon gas diffusing through the walls of the inner package(s). Thus, the inner package or packages may each have their own scavenger, and the larger outer container has a scavenger. Furthermore, the scavenger 18 of the larger outer airtight container 16 may be maintained independently without disturbing the sealed package or packages, and thus extending the shelf life of the packaged implants. The construction of the larger outer container may be similar to the package 12 or may have a different construction.

In an alternate arrangement, the package 12 may be formed from a gas breathable material and the larger outer airtight container 16 may include the scavenger, which then draws the carbon gas or gases from the package 12 into the larger outer airtight container 16.

In yet another embodiment, the implant or implants 10 may be stored in an interim container, such as a chamber, prior to sealing the implant in the package. For example, the implant may be placed in a container that is filled with an inert gas for a holding period prior to sealing in the package.

In yet another embodiment, the implant 10 may be placed in the package 12 but not sealed therein and then placed in a chamber where the air is removed, including air from the packaging. An inert gas may be directed or injected into the chamber, which could be repeated, i.e., gas removed and then injected, followed by removal of the package and then sealing the package with the implant contained therein.

Accordingly, a medical implant is described herein that is packaged and handled in accordance with one or more of any of the above described methods and/or treatments.

For example, it is believed that a medical implant that has been handled and/or stored in the packaging described above for less than one year but at least 120 days using one or more the above described methods, and optionally one or more of the post-formation treatments described herein, can retain at least 80% of hydrophilicity, 90% of hydrophilicity, or about 100% of hydrophilicity.

In another example, a medical implant is provided that has a medical implant body with a titanium implant surface with an initial water contact angle of less than 5 degrees immediately after formation and pre-packaging and a post packaging water contact angle, where the post packaging water contact angle covers a packaging period of less than 1 year and at least 120 days, and which post packaging water contact angle may be less than 30 degrees, optionally, less than 20 degrees, and optionally less than 10 degrees.

It is also believed that an implant that has been handled and/or stored in the packaging described above for less than three years will remain hydrophilic.

It is understood that the above-described embodiments are illustrative only. Numerous and various other arrangements can be made by those skilled in the art without departing from the spirit and scope of the disclosure. While specific embodiments and applications of the present disclosure have been illustrated and described, it is to be understood that the disclosure is not limited to the precise configuration and components disclosed herein. Various modifications, changes, and variations that will be apparent to those skilled in the art may be made in the arrangement, operation, and details of the methods and systems of the present disclosure disclosed herein without departing from the spirit and scope of the disclosure.

The embodiments of the invention in which an exclusive property or privilege is claimed are defined as follows:

1. A method of handling a medical implant, the implant comprising a body and having a titanium implant surface, said method comprising:
providing a sealable package;
placing the medical implant in the sealable package within 12 hours or 6 hours or about 2 hours after the implant has been formed;
sealing the medical implant in the sealable package to form a sealed package within 1 hour to 2 minutes after any last post-formation treatment and/or placing the medical implant in the sealable package; and
removing the air in the package prior to or while sealing the package to form a low organic or carbon atmosphere or organic-free or carbon gas-free atmosphere in the sealed package.

2. The method according to claim 1, further comprising removing the air contained inside the sealable package prior to said placing the medical implant in the sealable package.

3. The method according to claim 1, wherein said providing a sealable package includes providing a sealable package that is sufficiently robust to maintain the low organic or carbon atmosphere or organic-free or carbon gas-free atmosphere in the sealed package through the packaging processes, sterilization, and product storage, shipment, and delivery to an operating room.

4. The method according to claim 1, further comprising sterilizing the sealed package, wherein said providing a sealable package includes providing a sealable package that is sufficiently robust to maintain the low organic or carbon atmosphere or organic-free or carbon gas-free atmosphere in the sealed package through the sterilization process.

5. The method according to claim 1, wherein said providing a sealable package includes providing a sealable package that is capable of maintaining a low pressure during its time of handling and storage up until implantation.

6. The method according to claim 1, wherein said providing a sealable package includes providing a sealable package being formed from a heat sealable synthetic material, such as a material selected from the group consisting of Polyethylene (PE), Polyvinylidenchloride (PVDC), Polypropylene (PP), Polyvinylchloride (PVC), Polyester (PET), and Polyamide (PA), with a metal foil.

7. The method according to claim 1, further comprising including an absorbent or adsorbent to actively attract and bind carbon gas present in the sealable package to reduce and/or eliminate any available carbon gas within the sealed package.

8. The method according to claim 7, wherein said including an absorbent or adsorbent in the sealable package includes placing the absorbent or adsorbent in the sealable package prior to placing the medical implant in the sealable package.

9. The method according to claim 7, after sealing the implant and the absorbent or adsorbent in the sealable package and after a period of time after said sealing to allow the absorbent or adsorbent to reduce and/or eliminate carbon gas within the sealed package, further comprising sealing the absorbent or adsorbent in a portion of a heat sealed package to form a separate compartment from a main compartment enclosing the medical implant.

10. The method according to claim 9, further comprising removing the separate compartment from the main compartment of the sealed package without unsealing the main compartment.

11. The method according to claim 7, wherein said providing a sealable package includes providing a sealable package with a main compartment for holding the medical implant and a secondary compartment for holding the absorbent or adsorbent.

12. A method of handling a medical implant, the implant comprising a body and having a titanium implant surface, said method comprising:
providing a sealable package;
displacing organic material from the titanium implant surface;
placing the medical implant in the sealable package within 2 hours after displacing the organic material from the implant surface; and
sealing the implant in the sealable package to form a sealed package within about 60 to 2 minutes after placing the medical implant in the sealable package to form a low organic or carbon atmosphere or organic-free or carbon gas-free atmosphere in the sealed package.

13. The method according to claim 12, wherein said displacing includes exposing the medical implant to UV light.

14. The method according to claim 12, further comprising sterilizing the sealed package.

15. The method according to claim 14, further comprising including an absorbent or adsorbent to actively attract and bind carbon gas present in the sealable package to reduce and/or eliminate carbon gas within the sealed package, and removing said absorbent or adsorbent before said sterilizing.

16. The method according to claim 15 wherein said providing a sealable package includes providing a sealable package with a main compartment for holding the medical implant and a secondary compartment for holding the absorbent or adsorbent.

17. The method according to claim 16, wherein said providing a sealable package includes providing a sealable package with a removable secondary compartment, the secondary compartment being removable from the main compartment without unsealing the main compartment.

18. The method according to claim 12, further comprising applying a sodium chloride nano-layer after the formation process.

19. The method according to claim 12, further comprising storing the sealed package in a larger airtight container.

20. The method according to claim 19, wherein said storing the sealed package in a larger air tight container includes storing the sealed package in a larger air tight container with an absorbent or adsorbent contained therein, wherein the ambient atmosphere around the sealed package has an atmosphere with the organic material, such as carbon gas, removed.

21. The method according to claim 12, wherein prior to sealing the medical implant into the package, further comprising flushing the package with an inert gas or a carbon displacing gas.

22. The method according to claim 12, wherein prior to placing the medical implant into the package, further comprising flushing the package with an inert gas or a carbon displacing gas.

23. The method according to claim 12, wherein prior to placing the medical implant into the package and after placing the medical implant into the package further comprising flushing the package with an inert gas or a carbon displacing gas.

24. The method according to claim 21, wherein said flushing comprises flushing the package with Argon or Nitrogen gas or a carbon displacing gas.

25. A method of handling a medical implant, the implant comprising a body and having a titanium implant surface, said method comprising:
providing a sealable package;
placing the medical implant in the sealable package;
sealing the implant in the sealable package to form a sealed package; and
forming a low organic or carbon atmosphere or organic-free or carbon gas-free atmosphere in the sealed package sufficient to maintain a water contact angle of the implant surface of less than 30 degrees after 120 days after forming the implant surface.

26. The method according to claim 25, further comprising selecting an implant with a titanium implant surface, the titanium implant surface having a plurality of nanotubes formed therein with a water contact angle of less than 5 degrees, and optionally less than 1 degree.

27. The method according to claim 26, wherein said forming includes forming the low organic or carbon atmosphere or organic-free or carbon gas-free atmosphere in the sealed package sufficient to maintain a water contact angle of the nanotube surface of less than 30 degrees or 20 degrees after 120 days after forming the nanotube surface.

28. The method according to claim 26, wherein said forming includes forming the low organic or carbon atmosphere or organic-free or carbon gas-free atmosphere in the sealed package sufficient to maintain a water contact angle of the nanotube surface of less than 10 degrees after 120 days after forming the nanotube surface.

29. A method of handling a medical implant, the implant comprising a body and having a titanium implant surface, said method comprising:
providing a sealable package;
placing the medical implant in the sealable package;
sealing the implant in the sealable package to form a sealed package; and
forming a low organic or carbon atmosphere or organic-free or carbon gas-free atmosphere in the sealed package sufficient to maintain a water contact angle decay rate of less than 1/10th of a degree per day.

30. The method according to claim 25, further comprising including an absorber or adsorber to attract and bind carbon gas present in the sealable package to reduce and/or eliminate carbon gas within the sealed package.

31. The method according to claim 25, further comprising exposing the medical implant to UV light.

32. The method according to claim 25, wherein prior to sealing the medical implant into the package, further comprising flushing the package with an inert gas or a carbon displacing gas.

33. The method according to claim 12, further includes providing the titanium implant surface of the medical implant with a plurality of TiO2 nanotubes formed thereon.

34. A medical implant packaged in accordance with the method of claim 1.

35. A medical implant comprising:
a medical implant body with a titanium implant surface with an initial hydrophilicity immediately after formation and pre-packaging and a post packaging hydrophilicity, the post packaging hydrophilicity covering a packaging period of less than 1 year and at least 120 days, and said post packaging hydrophilicity being at least 80% of said initial hydrophilicity.

36. The medical implant of claim 35, wherein said post packaging hydrophilicity is at least 90% of said initial hydrophilicity.

37. The medical implant of claim 35, wherein said post packaging hydrophilicity is about 100% of said initial hydrophilicity.

38. A medical implant comprising:
a medical implant body with a titanium implant surface with an initial hydrophilicity immediately after formation and pre-packaging and a post packaging hydrophilicity, the post packaging hydrophilicity covering a packaging period of less than 1 year and at least 120 days, and said post packaging hydrophilicity being at least 80% of said initial hydrophilicity.

39. The medical implant of claim 38, wherein said post packaging water contact angle is less than 20 degrees.

40. The medical implant of claim 38, wherein said post packaging water contact angle is less than 10 degrees.

41. The medical implant of claim 38, wherein the medical implant is selected from one of a full joint replacement implant, a partial joint replacement implant, a plate, a catheter, a screw, a mesh, a stent, a housing, a spacer, a connector, a rod, a pin, a wire, a bone replacement, a tissue scaffold, a fixation device, a nail, a trans-cutaneous interface, and an anchor.

42. The medical implant of claim 38, wherein the medical implant is a temporary implant.

43. A medical implant comprising:
a medical implant body with a titanium implant surface having a hydrophilicity after a packaging period of at least three years so that a water drop measurement will show a water contact angle of less than 90 degrees.

44. A method of handling a medical implant, the implant comprising a body and having a titanium implant surface, said method comprising:
providing a sealable package;
placing the medical implant in the sealable package;
sealing the implant in the sealable package to form a sealed package; and
forming a low organic or organic-free atmosphere in the sealed package sufficient to allow the implant surface to retain hydrophilicity for at least three years so that a water drop measurement will show a water contact angle of less than 90 degrees.
